(19) United States
(12) Patent Application Publication
(10) Pub. No.: US 2023/0398048 A1
(43) Pub. Date: Dec. 14, 2023

(54) NOVEL SYSTEM FOR SOLUBILISING FAT-SOLUBLE ORGANIC SUN FILTERS

(71) Applicant: PIERRE FABRE DERMO-COSMETIQUE, BOULOGNE-BILLANCOURT (FR)

(72) Inventors: Hélène DROMIGNY, TOULOUSE (FR); Karine TOUITOU, COLOMIERS (FR); Roxane PROVOST, TOULOUSE (FR); Marie PLANTIE, LACROIX FALGARDE (FR)

(73) Assignee: PIERRE FABRE DERMO-COSMETIQUE, BOULOGNE-BILLANCOURT (FR)

(21) Appl. No.: 18/036,432

(22) PCT Filed: Nov. 10, 2021
(86) PCT No.: PCT/FR2021/051998

§ 371 Date: May 11, 2023

(30) Foreign Application Priority Data
Nov. 12, 2020 (FR) FR2011622

(51) Int. Cl. A61K 8/49; A61Q 17/04; A61K 8/41; A61K 8/35

(52) U.S. Cl. CPC A61K 8/4966; A61Q 17/04; A61K 8/415; A61K 8/35; A61K 2800/30; A61K 2800/412; A61K 2800/5922

(57) ABSTRACT

The present invention relates to a cosmetic or dermatological composition in the form of an emulsion, comprising: (a) a fat-soluble photoprotective system consisting of one or more fat-soluble UV filter(s) selected from: (i) hexyl 2-[4-(diethylamino)-2 hydroxybenzoyl]benzoate, (ii) 2,4-bis[4-(2-ethythexyloxy)-2-hydroxyphenyl]-6-(4-methoxyphenyl)-1,3,5-triazine, (iii) butyl methoxydibenzoylmethane, (iv) ethythexyl triazone, and (v) diethylhexyl butamido triazone: (b) a solubilising system of the fat-soluble photoprotective system (a), comprising C12-C15 alkyl benzoate in a quantity greater than or equal to 80% by weight, preferably greater than or equal to 90% by weight, relative to the total weight of the solubilising system; and (c) one or more particulate organic UV filters, which are in particular hydro-dispersed and are selected from: (vi) 5,6,5′,6′-tetraphenyl-3,3′-(1,4-phenylene)-bis[1,2,4]triazine, (vii) methylene bis-benzotriazolyl tetramethylbutylphenol, (viii) tris-biphenyl triazine; characterised in that: —the fat-soluble photoprotective system (a) constitutes between 3% and 19% by weight, preferably between 5% and 15% by weight, relative to the total weight of the composition; —and —the solubilising system (b) constitutes between 15% and 30% by weight, relative to the total weight of the composition; the composition not comprising ethylhexyl salicylate, octocrylene, ethylhexyl methoxycinnamate, isoamyl methoxylcinnamate, homosalate, para-aminobenzoic acid (PABA), octyl dimethyl PABA, 3-methylbenzylidene 30 camphor, 4-methylbenzylidene camphor, benzophenone-3 and/or benzophenone-4.

### FIELD OF THE INVENTION

The present invention relates to a cosmetic or dermatological composition in the form of an emulsion comprising a fat-soluble photoprotective system, a system for solubilising said fat-soluble photoprotective system, and one or more, in particular hydro-dispersed, particulate organic UV filters.

### PRIOR ART

The deleterious effects of ultraviolet radiation on the skin are well-known, and a multitude of sun protection products, commonly called sun-care products, are marketed. These sun-care products, intended for topical application, comprise a combination of fat-soluble, water-soluble or non-soluble, organic or inorganic compounds, which protect the user from UV radiation and, for this reason, are qualified as sun filters.

The need to combine a plurality of sun filters within a given composition is explained by the fact that each filter preferentially absorbs certain wavelengths. Therefore, in order to effectively protect the user against all ultraviolet radiation, it is necessary to combine a plurality of filters in order to obtain the broadest possible UV absorption spectrum.

However, certain liquid sun filters, including ethylhexyl salicylate or octocrylene are known to facilitate the solubilising of fat-soluble organic filters, the sun filters most used in sun protection products, a number of which are initially in powder form.

However, octocrylene, as well as ethylhexyl salicylate, are suspected of being potential endocrine disruptors. Other sun filters present in many commercial products are also subject to controversy, because of their potentially harmful effects on the user or for the environment. These include, in particular, derivatives of cinnamate (ethylhexyl methoxycinnamate, isoamyl methoxylcinnamate), homosalate, para-aminobenzoic acid (PABA, octyl dimethyl PABA), camphor (3-methylbenzylidene camphor, 4-methylbenzylidene camphor) benzophenone (benzophenone-3, benzophenone-4). The use of such compounds is therefore undesirable.

Furthermore, more generally, it is desirable to minimise both the number of ultraviolet filters forming a photoprotective composition, as well as the concentration of said filters within the composition, in order to obtain better results in terms of user tolerance and environmental impact. Indeed, not only are not all organic chemical filters perfectly harmless per se, but the risk of harmful interactions between the various combined compounds cannot be ignored. The minimisation of the number and concentration of filters within sun-care products is in addition beneficial for the environment, to the extent that UV filters contained in a sunscreen can end up in the ocean.

Consequently, the Applicant, which places protection of the user and the environment at the centre of its concerns in the development of new sun-care products, has refrained from using sun filters when their harmlessness is in doubt.

The fact remains that it is necessary to perfectly solubilise the various filters constituting the sun-care composition, in particular the fat-soluble filters present in powder form, in order to obtain a homogeneous composition that is stable over time, so as to guarantee effective protection from UV radiation.

In addition to its safety, the Applicant has wished to ensure the comfort of the user by proposing a sun-care product with optimum sensory feel, which is in particular easy to spread, and which once applied, does not give a greasy or sticky appearance to the skin, whether to the touch or visually.

### Technical Problem

The Applicant has therefore sought to develop novel sun-protection compositions efficiently protecting the user from UV radiation, comprising a novel solubilising system which can efficiently solubilise the fat-soluble UV filters in powder form, while having an optimum sensory feel. It should be noted that the concept of efficiency of the solubilising system also covers the stability of the solution obtained by the solubilisation of fat-soluble UV filters in the solubilising system, within which the filters must not recrystallise over time.

Surprisingly and unexpectedly, the Applicant has found a novel use for C₁₂-C₁₅ alkyl benzoate as an agent for solubilising fat-soluble UV filters in a composition in emulsion form, which provides the desired properties in terms of sun protection while offering a sensorially improved galenic form.

Furthermore, in contrast to the majority of sun-care products currently on the market, the composition developed by the Applicant is characterised by very good results obtained during in vitro ocular tolerance tests. This is a considerable advantage, since it is frequently the case that sun-care compositions come into contact with the eyes of a user, due to sweating or following a swim.

### SUMMARY OF THE INVENTION

According to a first aspect, the present invention therefore relates to a cosmetic or dermatological composition in the form of an emulsion, comprising:
(a) a fat-soluble photoprotective system, consisting of one or more fat-soluble UV filters chosen from:
(i) hexyl 2-[4-(diethylamino)-2-hydroxybenzoyl]benzoate,
(ii) 2,4-bis[4-(2-ethylhexyloxy)-2-hydroxyphenyl]-6-(4-methoxyphenyl)-1,3,5-triazine, (iii) butyl methoxydibenzoylmethane,
(iv) ethylhexyl triazone, and
(v) diethylhexyl butamido triazone;
(b) C₁₂-C₁₅ alkyl benzoate; and
(c) one or more, in particular hydro-dispersed, particulate organic UV filters, chosen from:
(vi) 5,6,5′,6′-tetraphenyl-3,3′-(1,4-phenylene)-bis[1,2,4]triazine, (vii) methylene bis-benzotriazolyl tetramethylbutylphenol, and
(viii) tris-biphenyl triazine;
and is characterised in that:
   the fat-soluble photoprotective system (a) constitutes between 3 wt % and 19 wt %, especially between 5 wt % and 19 wt %, in particular between 7 wt % and 17 wt %, typically between 9 wt % and 15 wt %, for example between 10 wt % and 13 wt % of the composition relative to the total weight of the composition; and
   C₁₂-C₁₅ alkyl benzoate (b) constitutes between 15 wt % and 30 wt %, especially between 20 wt % and 30 wt %, typically between 20 wt % and 25 wt %, relative to the total weight of the composition;
said composition not comprising ethylhexyl salicylate, octocrylene, ethylhexyl methoxycinnamate, isoamyl methoxylcinnamate, homosalate, para-aminobenzoic acid (PABA), octyl dimethyl PABA, 3-methylbenzylidene camphor, 4-methylbenzylidene camphor, benzophenone-3 and/or benzophenone-4.

According to a second aspect, the present invention relates to a cosmetic or dermatological composition in the form of an emulsion, comprising:
(a) a fat-soluble photoprotective system, consisting of one or more fat-soluble UV filters chosen from the filters (i) to (v);
(b) a solubilising system of said fat-soluble photoprotective system (a), comprising C₁₂-C₁₅ alkyl benzoate in a quantity greater than or equal to 80 wt %, preferably greater than or equal to 90 wt %, relative to the total weight of the solubilising system; and
(c) one or more, in particular hydro-dispersed, particulate organic UV filters, chosen from the filters (vi) to (viii);
characterised in that:
   the fat-soluble photoprotective system (a) constitutes between 3 wt % and 19 wt %, preferably between 5 wt % and 15 wt %, relative to the total weight of the composition; and
   the solubilising system (b) constitutes between 15 wt % and 30 wt %, relative to the total weight of the composition;
said composition not comprising ethylhexyl salicylate, octocrylene, ethylhexyl methoxycinnamate, isoamyl methoxylcinnamate, homosalate, para-aminobenzoic acid (PABA), octyl dimethyl PABA, 3-methylbenzylidene camphor, 4-methylbenzylidene camphor, benzophenone-3 and/or benzophenone-4.

### DETAILED DESCRIPTION

For the purposes of the present invention, “emulsion” shall mean any type of emulsion obtained by the dispersion of a discontinuous inner phase in a continuous outer phase, one of these phases being an aqueous phase and the other being an oily phase. It is, for example, an oil-in-water or water-in-oil or multiple emulsion, preferably an oil-in-water emulsion.

These emulsions can be more or less fluid and be, in particular, in the form of a white or coloured cream, an ointment, a milk, a lotion, a serum, a paste, or optionally an aerosol, a foam or a spray, and can also be water-resistant.

The composition according to the invention is intended for topical application, in particular on the skin and/or the hair.

For the purposes of the present invention, “photoprotective system” shall mean a combination of compounds which can, after application on a surface (skin, hair, etc.), by mechanisms of absorption and/or reflection and/or diffusion of UVA and/or UVB radiation, prevent, or at least limit, the contact of said radiation with said surface.

The compounds constituting the photoprotective system are called sun filters or UV filters.

The term “ultraviolet radiation” or “UV radiation”, commonly abbreviated as “UV”, designates solar ultraviolet radiation, but also artificial ultraviolet radiation generated by tanning lamps, for example.

The term “UVA radiation” or simply “UVA”, shall mean ultraviolet radiation having a wavelength, between 320 and 400 nm.

The term “UVB radiation” or simply “UVB”, shall mean ultraviolet radiation having a wavelength, between 290 and 320 nm.

The term “sun filter” or “UV filter” designates a substance capable of filtering UV radiation in order to protect a surface, typically the skin or hair, against the harmful effects of this radiation.

A sun filter is qualified as a “UVA filter” or “UVB filter” depending on whether it filters mostly UVA or UVB.

A sun filter referred to as “broad band” or “broad spectrum” filters both UVA and UVB.

According to the radiation filtering mechanism, in other words according to whether the radiation is absorbed and/or reflected, or even scattered by the sun filters, it is possible to distinguish, with complete rigour, between:
the stricto sensu filters, which mostly absorb radiation, and
screens, which absorb and reflect radiation.

However, in the remainder of the description, the term “sun filter”, sometimes abbreviated to “filter”, will be used interchangeably, whether it refers to a filter or a screen.

A sun filter can be “fat-soluble” or “water-soluble”, depending on whether it is solubilised more easily in lipids or in water, or can even be “non-soluble”. A non-soluble sun filter is typically in particulate form, and can nevertheless be incorporated in a sun-protection composition, especially in a dispersed form, especially hydro-dispersed, when it is dispersed in an aqueous phase.

The fat-soluble photoprotective system (a) that constitutes a composition according to the invention only comprises fat-soluble sun filters, and constitutes between 3 wt % and 19 wt %, preferably between 5 wt % and 15 wt %, relative to the total weight of the composition.

In a particular embodiment, the fat-soluble photoprotective system constitutes between 3 wt % and 19 wt %, especially between 5 wt % and 19 wt %, in particular between 7 wt % and 17 wt %, typically between 9 wt % and 15 wt %, for example between 10 wt % and 13 wt %, relative to the total weight of the composition.

The fat-soluble photoprotective system that constitutes a composition according to the invention, consists of one or more fat-soluble UV filters chosen from the following filters (i) to (v):
(i) hexyl 2-[4-(diethylamino)-2-hydroxybenzoyl]benzoate (CAS Number: 302776-68-7), also called diethylamino hydroxybenzoyl hexyl benzoate or DHHB, marketed by BASF under the name Uvinul A Plus*.

This is a fat-soluble UVA filter, having a maximum absorption wavelength λₘₐₓ equal to 354 nm.

In a particular embodiment, DHHB constitutes between 1 wt % and 10 wt %, for example between 3 wt % and 10 wt %, in particular between 4 wt % and 9 wt %, typically between 5 wt % and 8 wt %, in particular between 5 wt % and 7 wt %, especially between 6 wt % and 7 wt % or advantageously between 5 wt % and 6 wt %, relative to the total weight of the composition.
(ii) 2,4-bis[4-(2-ethylhexyloxy)-2-hydroxyphenyl]-6-(4-methoxyphenyl)-1,3,5-triazine (CAS Number: 187393-00-6), also called bis-ethylhexyloxyphenol methoxyphenyl triazine or BEMT, marketed by BASF under the name Tinosorb S©.

This is a broad band fat-soluble filter, having two absorption peaks at 310 nm and 340 nm.

In a particular embodiment, BEMT constitutes between 1 wt % and 4 wt %, especially between 2 wt % and 4 wt %, in particular between 2 wt % and 3 wt % relative to the total weight of the composition.
(iii) 4-tert-butyl-4-methoxydibenzoylmethane or butyl methoxydibenzoylmethane (chemical name: 1,4-(1,1-dimethylethyl)phenyl-3-(4-methoxyphenyl)-1,3-propanedione; CAS Number: 70356-09-1), also called avobenzone or BMDBM, marketed, especially, by DSM under the name Parsol 1789©.

This is a fat-soluble UVA filter, having a maximum absorption wavelength λₘₐₓ equal to 357 nm.

In a particular embodiment, BMDBM constitutes between 1 wt % and 10 wt %, for example between 1 wt % and 8 wt %, in particular between 1 wt % and 5 wt %, typically between 2 wt % and 4 wt %, relative to the total weight of the composition. (iv) ethylhexyl triazone or EHT (chemical name: 4-[[4,6-bis[[4-(2-ethylhexoxy-oxomethyl)phenyl]amino]-1,3,5-triazin-2-yl]amino]benzoic acid 2-ethylhexyl ester; CAS Number: 88122-99-00), marketed by BASF under the name Uvinul T 150®.

This is a fat-soluble UVB filter, having a maximum absorption wavelength λₘₐₓ equal to 314 nm.

In a particular embodiment, EHT constitutes between 1 wt % and 6 wt %, typically between 2 wt % and 5 wt %, especially between 3 wt % and 5 wt %, in particular between 3.5 wt % and 4.5 wt % relative to the total weight of the composition.
(v) diethylhexyl butamido triazone or DBT (chemical name: 4,4′-[[6-[[4-[[(1,1-dimethylethyl)amino]carbonyl]phenyl]amino]-1,3,5-triazine-2,4-diyl]diimino]bis-, bis(2-ethylhexyl)benzoate; CAS Number: 154702-15-5), marketed by 3V Sigma under the name Uvasorb HEB®.

This is a fat-soluble UVB filter, having a maximum absorption wavelength λₘₐₓ equal to 310 nm.

In a particular embodiment, DBT constitutes between 1 wt % and 6 wt %, typically between 2 wt % and 5 wt %, especially between 3 wt % and 5 wt %, in particular between 3.5 wt % and 4.5 wt % relative to the total weight of the composition.

The fat-soluble photoprotective system (a) may consist of a single fat-soluble filter chosen from the filters (i) to (v).

Preferably however, it consists of the combination of at least two fat-soluble filters, the first being chosen from filters (i) to (iii) and the second from filters (iv) and (v), the second filter being in particular filter (iv). In particular, the fat-soluble photoprotective system (a) consists of the combination of three filters, the first being chosen from filters (i) or (iii), the second from filters (iv) and (v), and the third filter corresponding to filter (ii), the second filter being advantageously filter (iv).

In a particular embodiment, the fat-soluble photoprotective system (a) consists of the following combinations of fat-soluble filters:
(a1) hexyl 2-[4-(diethylamino)-2-hydroxybenzoyl]benzoate, 2,4-bis[4-(2-ethylhexyloxy)-2-hydroxyphenyl]-6-(4-methoxyphenyl)-1,3,5-triazine and ethylhexyl triazone;
(a2) hexyl 2-[4-(diethylamino)-2-hydroxybenzoyl]benzoate, 2,4-bis[4-(2-ethylhexyloxy)-2-hydroxyphenyl]-6-(4-methoxyphenyl)-1,3,5-triazine and diethylhexyl butamido triazone;
(a3) 2,4-bis[4-(2-ethylhexyloxy)-2-hydroxyphenyl]-6-(4-methoxyphenyl)-1,3,5-triazine and ethylhexyl triazone;
(a4) 2,4-bis[4-(2-ethylhexyloxy)-2-hydroxyphenyl]-6-(4-methoxyphenyl)-1,3,5-triazine and diethylhexyl butamido triazone;
(a5) hexyl 2-[4-(diethylamino)-2-hydroxybenzoyl]benzoate, butyl methoxydibenzoylmethane, 2,4-bis[4-(2-ethylhexyloxy)-2-hydroxyphenyl]-6-(4-methoxyphenyl)-1,3,5-triazine and ethylhexyl triazone;
(a6) hexyl 2-[4-(diethylamino)-2-hydroxybenzoyl]benzoate, butyl methoxydibenzoylmethane, 2,4-bis[4-(2-ethylhexyloxy)-2-hydroxyphenyl]-6-(4-methoxyphenyl)-1,3,5-triazine and diethylhexyl butamido triazone;
(a7) butyl methoxydibenzoylmethane, 2,4-bis[4-(2-ethylhexyloxy)-2-hydroxyphenyl]-6-(4-methoxyphenyl)-1,3,5-triazine and ethylhexyl triazone; and
(a8) butyl methoxydibenzoylmethane, 2,4-bis[4-(2-ethylhexyloxy)-2-hydroxyphenyl]-6-(4-methoxyphenyl)-1,3,5-triazine and diethylhexyl butamido triazone.

In particular, the fat-soluble photoprotective system (a) consists of one of the combinations (a1), (a3), (a5) or (a7).

When the fat-soluble photoprotective system (a) corresponds to one of the combinations of fat-soluble filters (a1)-(a8), this constitutes between 3 wt % and 19 wt %, preferably between 5 wt % and 15 wt %, relative to the total weight of the composition.

In a particular embodiment, the fat-soluble photoprotective system corresponds to one of the combinations of fat-soluble filters (a1)-(a8), which constitutes between 3 wt % and 19 wt %, especially between 5 wt % and 19 wt %, in particular between 7 wt % and 17 wt %, typically between 9 wt % and 15 wt %, for example between 10 wt % and 13 wt % of the composition relative to the total weight of the composition.

In particular, the fat-soluble photoprotective system (a) consists of the combination (a1) or (a2).

In this particular embodiment, DHHB constitutes between 1 wt % and 10 wt %, for example between 3 wt % and 10 wt %, in particular between 4 wt % and 9 wt %, typically between 5 wt % and 8 wt %, for example between 5 wt % and 7 wt %, especially between 6 wt % and 7 wt % or advantageously between 5 wt % and 6 wt %, relative to the total weight of the composition, BEMT constitutes between 1 wt % and 4 wt %, especially between 2 wt % and 4 wt %, in particular between 2 wt % and 3 wt % of the composition relative to the total weight of the composition, and EHT or DBT constitutes between 1 wt % and 6 wt %, typically between 2 wt % and 5 wt %, especially between 3 wt % and 5 wt %, in particular between 3.5 wt % and 4.5 wt % relative to the total weight of the composition.

In an advantageous embodiment, the fat-soluble photoprotective system (a) consists of the combination of fat-soluble filters (a1).

The fat-soluble photoprotective system (a) is solubilised in a solubilising system (b).

For the purposes of the present invention, the term “solubilising system”, being understood solubilising the fat-soluble photoprotective system (a) as described above, designates a liquid lipophilic compound or a combination of a plurality of liquid lipophilic compounds, typically a liquid lipophilic emollient or a combination of liquid lipophilic emollients, especially a liquid fatty substance or a combination of liquid fatty substances, capable to totally solubilising the one on more fat-soluble UV filters (i) to (v), thus making it possible to obtain a transparent homogeneous solution, the one or more fat-soluble UV filters (i) to (v) being solubilised in a quantity or quantities such that the weight ratio of the one or more fat-soluble UV filters (i) to (v)/solubilising system is greater than or equal to 0.2, typically at least equal to 0.3, preferably at least equal to 0.4, and especially less than 0.7, in particular less than or equal to 0.6.

The term “lipophilic liquid compound” shall mean a compound which has an affinity for apolar substances such as lipids, and is in liquid form especially for a temperature between 5° C. and ambient temperature.

For the purposes of the present invention, “ambient temperature” shall mean a temperature between 15° C. and 25° C., preferably between 20° C. and 25° C.

The term “homogeneous solution” shall designate the absence of crystals and/or aggregates visible to the naked eye, in the solution.

The term “transparent solution” shall designate a clear solution, in other words a solution which has no cloudiness.

This ability to solubilise the one or more fat-soluble UV filters (i) to (v) is evaluated in two steps.

In a first step, the one or more fat-soluble UV filters (i) to (v) are introduced into the solubilising system (b) at the temperature of preparation of the oily phase of the emulsion according to the invention, typically at 80° C., while stirring, in order to solubilise it or them.

A clear homogeneous solution must be obtained in a period less than 5 hours, especially less than 4 hours, in particular less than 2 hours, and preferably less than 1 hour. The homogeneous transparent solution thus obtained is then cooled to ambient temperature, or to 5° C., in the presence or absence of stirring.

After solubilisation, typically carried out under the conditions described above, the solubilising system according to the invention makes it possible to maintain the one or more fat-soluble UV filters (i) to (v) in solubilised form, in other words it prevents even partial recrystallisation of the one more fat-soluble UV filters (i) to (v).

Thus, once the homogeneous solution is obtained by the solubilisation of the one or more fat-soluble UV filters (i) to (v) in the solubilising system (b), it remains homogeneous and clear, in the presence or absence of stirring, at a temperature of 5° C. and at ambient temperature, for at least 24 hours, preferably at least 1 week, in particular at least 1 month.

In a preferred embodiment, the solution obtained by the solubilisation of the one or more fat-soluble UV filters (i) to (v) in the solubilising system (b) remains homogeneous and clear, in the presence or absence of stirring, at ambient temperature and advantageously at a temperature of 5° C., for at least 6 months, preferably at least 12 months, especially at least 24 months, in particular at least 36 months.

The solubilising system (b) according to the invention comprises C₁-C₁₅ alkyl benzoate in a quantity greater than or equal to 80 wt %, preferably greater than or equal to 90 wt %, advantageously greater than or equal to 95 wt %, especially greater than or equal to 98 wt %, in particular greater than or equal to 99 wt %, relative to the total weight of the solubilising system.

According to a particular embodiment, C₁₂-C₁₅ alkyl benzoate constitutes 100 wt % relative to the total weight of the solubilising system.

For the purposes of the present invention, “C₁₂-C₁₅ alkyl benzoate” shall mean a compound of general formula C₆H₅—C(O)OR, in which R represents a (C₁₂-C₁₅)-alkyl group, or a mixture of these.

For the purposes of the present invention, “(C₁₂-C₁₅)alkyl” shall mean a monovalent saturated, linear or branched, hydrocarbon chain having 12 to 15 carbon atoms.

The term “C₁₂-C₁₅ alkyl benzoate” refers in particular to the following commercial products: Cetiol AB© (BASF), Crodamol AB© (Croda), Finsolv TN® (Innospec) and Tegosoft TN® (Evonik).

As is apparent from the above, a person skilled in the art will be able to verify that the combination of one or more liquid lipophilic compounds, typically of one or more liquid lipophilic emollients, with C₁₂-C₁₅ alkyl benzoate as the majority component, which constitutes at least 80 wt %, preferably at least 90 wt %, advantageously at least 95 wt %, especially at least 98 wt %, in particular at least 99 wt %, especially 100 wt % relative to the total weight of said combination, constitutes a solubilising system within the meaning of the present invention, by applying the following protocol:
introducing the fat-soluble photoprotective system according to the invention, as described above, into the solubilising system, in a weight ratio fat-soluble system/solubilising system greater than or equal to 0.2, typically at least equal to 0.3, preferably at least equal to 0.4, and especially less than 0.7, in particular less than or equal to 0.6;
placing the mixture obtained while stirring, preferably while stirring mechanically by means of a magnetic bar, at a temperature between 70° C. and 85° C., typically at 80° C., and continuing to stir for a period between 5 minutes and 5 hours, especially between 10 minutes and 4 hours, in particular between 20 minutes and 2 hours, typically between 30 minutes and 1 hour;
observing the obtaining of a homogeneous transparent solution, in other words the absence of cloudiness and crystals and/or aggregate visible to the naked eye in the solution;
cooling the homogeneous solution to ambient temperature, in other words typically to a temperature between 15° C. and 25° C., preferably between 20° C. and 25° C., in the presence or absence of stirring;
leaving the solution to rest as is for at least 24 hours, in particular at least 1 week and up to 3 months, typically for 1 month; and
observing the maintenance of the homogeneity of the solution, in other words the absence of crystals and/or aggregates visible to the naked eye in the solution.

Alternatively, after obtaining a homogeneous transparent solution, the solution can be cooled to approximately 5° C., typically to 5° C., and be left to rest in the absence of stirring for at least 24 hours, in particular at least 1 week and up to 1 month, in order to then observe the maintenance of the homogeneity of the solution.

The one or more liquid lipophilic compounds being able to be combined with C₁₂-C₁₅ alkyl benzoate in order to form the solubilising system according to the invention, typically comprise one or more polar chemical functions chosen from the functions: ester (—CO₂—), alcohol (—OH), ether (—O—), carbonate (—OCO₂—) and carboxylic acid (—CO₂H), and one or more lipophilic groups, in other words one or more aliphatic and/or aromatic hydrocarbon groups, in particular one or more saturated or unsaturated, linear or branched hydrocarbon chains, having between 6 and 20 carbon atoms, for example between 7 and 18 carbon atoms, especially between 8 and 16 carbon atoms, a phenyl group (—C₆H₅) and/or a phenylalkyl group.

In a particular embodiment, the one or more lipophilic compounds, especially the one or more liquid lipophilic emollients that can be combined with C₁₂-C₁₅ alkyl benzoate in order to form the solubilising system according to the invention, are chosen from: caprylic/capric triglycerides, dicaprylyl carbonate, coco caprylate, diisopropyl sebacate, diethylhexyl succinate, isopropyl palmitate, isopropyl myristate, propylheptyl caprylate, phenethyl benzoate, dibutyl adipate, diisopropyl adipate, glyceryl tri-2-ethylhexanoate, pentaerythrityl tetra-2-ethylhexanoate, cetyl 2-ethylhexanoate, isononyl isononanoate, butylene glycol dicaprylate/dicaprate, octyldodecanol, propylene glycol dicaprylate/dicaprate and dicaprylyl ether, in particular from caprylic/capric triglycerides, dicaprylyl carbonate, isopropyl palmitate, propylheptyl caprylate, phenethyl benzoate, diisopropyl adipate, glyceryl tri-2-ethylhexanoate, pentaerythrityl tetra-2-ethylhexanoate, cetyl 2-ethylhexanoate, isononyl isononanoate, butylene glycol dicaprylate/dicaprate, octyldodecanol, propylene glycol dicaprylate/dicaprate and dicaprylyl ether.

In a particular embodiment, the solubilising system (b) is formed from one or more liquid lipophilic emollients chosen from C₁₂-C₁₅ alkyl benzoate, caprylic/capric triglycerides and dicaprylyl carbonate, preferably from C₁₂-C₁₅ alkyl benzoate and caprylic/capric triglycerides, C₁₂-C₁₅ alkyl benzoate constituting a quantity greater than or equal to 80 wt %, preferably greater than or equal to 90 wt %, advantageously greater than or equal to 95 wt %, especially greater than or equal to 98 wt %, in particular greater than or equal to 99 wt %, especially 100 wt %, relative to the total weight of the solubilising system.

The solubilising system (b) constitutes between 15 wt % and 30 wt %, especially between 20 wt % and 30 wt %, typically between 20 wt % and 25 wt %, relative to the total weight of the composition.

In particular, C₁₂-C₁₅ alkyl benzoate constitutes between 15 wt % and 30 wt %, especially between 20 wt % and 30 wt %, typically between 20 wt % and 25 wt %, relative to the total weight of the composition.

In a particular embodiment of a composition according to the invention, the fat-soluble photoprotective system (a) is as described above, and the weight ratio fat-soluble photoprotective system (a)/solubilising system (b) is greater than or equal to 0.2, typically greater than or equal to 0.3, especially greater than or equal to 0.4, advantageously greater than or equal to 0.5, in particular strictly greater than 0.5, and especially less than 0.7, typically less than or equal to 0.6, in particular less than or equal to 0.55.

In particular, the fat-soluble photoprotective system (a) is as described above, and the weight ratio fat-soluble photoprotective system (a)/C₁₂-C₁₅ alkyl benzoate is greater than or equal to 0.2, typically greater than or equal to 0.3, especially greater than or equal to 0.4, advantageously greater than or equal to 0.5, in particular strictly greater than 0.5, and especially less than 0.7, typically less than or equal to 0.6, in particular less than or equal to 0.55.

The fat-soluble photoprotective system (a) constitutes between 3 wt % and 19 wt %, especially between 5 wt % and 19 wt %, in particular between 7 wt % and 17 wt %, typically between 9 wt % and 15 wt %, for example between 10 wt % and 13 wt % of the composition relative to the total weight of the composition, and consists in particular of one of the combinations (a1)-(a8), especially of one of the combinations (a1), (a3), (a5) or (a7), advantageously of the combination (a1).

The solubilising system (b) constitutes between 15 wt % and 30 wt %, especially between 20 wt % and 30 wt %, typically between 20 wt % and 25 wt %, relative to the total weight of the composition, and is in particular formed of one or more liquid lipophilic emollients chosen from C₁₂-C₁₅ alkyl benzoate, caprylic/capric triglycerides and dicaprylyl carbonate, preferably from C₁₂-C₁₅ alkyl benzoate and caprylic/capric triglycerides, C₁₂-C₁₅ alkyl benzoate constituting a quantity greater than or equal to 80 wt %, preferably greater than or equal to 90 wt %, advantageously greater than or equal to 95 wt %, especially greater than or equal to 98 wt %, in particular greater than or equal to 99 wt %, especially 100 wt %, relative to the total weight of the solubilising system.

In a particular embodiment, the composition according to the invention does not comprise diisopropyl sebacate, diethylhexyl succinate, isopropyl myristate and/or dibutyl adipate.

In another particular embodiment, the composition according to the invention does not comprise dicaprylyl carbonate, coco caprylate, diisopropyl sebacate, diethylhexyl succinate, isopropyl palmitate, isopropyl myristate, propylheptyl caprylate, phenethyl benzoate, dibutyl adipate, diisopropyl adipate, glyceryl tri-2-ethylhexanoate, pentaerythrityl tetra-2-ethylhexanoate, cetyl 2-ethylhexanoate, isononyl isononanoate, butylene glycol, dicaprylate/dicaprate, octyldodecanol, propylene glycol dicaprylate/dicaprate and/or dicaprylyl ether.

The composition according to the invention further comprises (c) one or more, in particular hydro-dispersed, particulate organic UV filters, chosen from the following filters (vi) to (viii).
(vi) 5,6,5′,6′-tetraphenyl-3,3′-(1,4-phenylene)-bis[1,2,4]triazine, (CAS Number: 55514-22-2) also called phenylene bis-diphenyltriazine.

This is mostly a non-soluble UVA filter, having a maximum absorption wavelength λₘₐₓ equal to 355 nm. It is, in particular, a hydro-dispersible filter.

Phenylene bis-diphenyltriazine is typically in the form of an aqueous dispersion, comprising between 20 wt % and 50 wt %, in particular between 40 wt % and 50 wt % active material relative to the total weight of the dispersion.

Advantageously, the method for preparing of the aqueous dispersion consists of grinding the insoluble organic filter in the form of particles, using a grinding apparatus and in the presence of a grinding aid.

Conventionally, wet grinding methods are used (wet grinding, wet mixing) and the grinding aid enables a better particle dispersion. These techniques are well-known to a person skilled in the art.

The grinding apparatus may be, for example, a microbead mill, a vibrating mill or a ball mill.

Depending on the grinding method, the grinding aid is chosen from the group consisting of anionic, non-ionic or amphoteric surfactants, emulsifiers and dispersants, such as PPG-1-PEG-9 Lauryl Glycol Ether (Eumulgin® L, marketed by BASF).

In a particular embodiment, phenylene bis-diphenyltriazine constitutes between 1 wt % and 5 wt %, especially between 2 wt % and 5 wt %, in particular between 2 wt % and 4 wt %, typically between 3 wt % and 4 wt % relative to the total weight of the composition.

It is understood that these weight percentages, as well as all of those indicated in the description, are expressed as weight percentages of active material relative to the total weight of the composition.

In a particular embodiment, the particle size D₅₀ of phenylene bis-diphenyltriazine is between 100 and 1000 nm, more particularly between 100 and 500 nm, yet more particularly between 120 and 400 nm, especially between 120 and 250 nm, in particular between 120 and 200 nm, typically between 150 and 200 nm.

The terms “D₅₀ size” or “median” shall mean the size for which the cumulative function F(D) is equal to 50 wt %, F(D) being defined according to the following equation:

$$F ( {D}_{i} ) = {\int }_{0}^{{D}_{i}} f ( D ) dD$$

In which:
f(D) is the particle size distribution by number, and
Dᵢ is a size class.

The value of D₅₀ will be determined according to the method described in the examples of the present description.
(vii) methylene bis-benzotriazolyl tetramethylbutylphenol (chemical name: 2,2′-methylene-bis-(6-(2H-benzotriazol-2-yl)-4-(1,1,3,3-tetramethylbutyl)phenol; CAS Number: 103597-45-1) also called MBBT, marketed by BASF under the name Tinosorb M®.

This is a non-soluble broad band filter, having two absorption peaks at 306 nm and 348 nm.

MBBT is typically present in the form of an aqueous dispersion, comprising between 40 wt % and 60 wt %, typically 50 wt % active material relative to the total weight of the dispersion.

In a particular embodiment, MBBT constitutes between 1 wt % and 7 wt %, typically 2 wt % and 6 wt %, especially between 2 wt % and 5 wt %, in particular between 3 wt % and 4 wt % relative to the total weight of the composition.

In a particular embodiment, the particle size D₅₀ of MBBT is between 50 nm and 250 nm, in particular between 60 nm and 150 nm.
(viii) 2,4,6-tris(biphenyl-4-yl)-1,3,5-triazine (CAS Number: 31274-51-8) also tris-biphenyl triazine or TBPT, marketed by BASF under the name Tinosorb A2B®.

This is mostly a non-soluble UVB filter, having a maximum absorption wavelength λₘₐₓ equal to 310 nm.

TBPT is typically in the form of an aqueous dispersion, comprising between 40 wt % and 60 wt %, typically 50 wt % active material relative to the total weight of the dispersion.

In a particular embodiment, TBPT constitutes between 1 wt % and 7 wt %, in particular between 2 wt % and 7 wt %, especially between 2 wt % and 6 wt % relative to the total weight of the composition.

In a particular embodiment, the particle size D₅₀ of TBPT is between 50 nm and 250 nm, in particular between 80 nm and 150 nm.

In a particular embodiment of the composition according to the invention, the one or more, in particular hydro-dispersed, particulate organic UV filters are chosen from phenylene bis-diphenyltriazine (vi) and MBBT (vii).

Advantageously, the composition according to the invention comprises a single particulate organic UV filter, preferably phenylene bis-diphenyltriazine.

According to a particular embodiment of the composition according to the invention, the weight ratio particulate organic UV filter(s) (c)/fat-soluble photoprotective system (a) is less than or equal to 0.35, in particular less than or equal to 0.33, advantageously less than or equal to 0.32, especially less than or equal to 0.30, and especially greater than or equal to 0.2.

According to a particular embodiment, the cosmetic or dermatological composition according to the invention comprises:
(a) a fat-soluble photoprotective system, consisting of one or more fat-soluble UV filters chosen from the filters (i) to (v);
(b) a solubilising system of said fat-soluble photoprotective system (a), comprising C₁₂-C₁₅ alkyl benzoate in a quantity greater than or equal to 80 wt %, preferably greater than or equal to 90 wt %, relative to the total weight of the solubilising system; and
(c) phenylene bis-diphenyltriazine as hydro-dispersed particulate organic UV filter, advantageously in a quantity between 1 wt % and 5 wt %, especially between 2 wt % and 5 wt %, in particular between 2 wt % and 4 wt %, typically between 3 wt % and 4 wt % relative to the total weight of the composition, and is characterised in that:
   the fat-soluble photoprotective system (a) constitutes between 3 wt % and 19 wt %, especially between 5 wt % and 19 wt %, in particular between 7 wt % and 17 wt %, typically between 9 wt % and 15 wt %, for example between 10 wt % and 13 wt % of the composition relative to the total weight of the composition; and
   the solubilising system (b) constitutes between 15 wt % and 30 wt %, relative to the total weight of the composition;
said composition not comprising ethylhexyl salicylate, octocrylene, ethylhexyl methoxycinnamate, isoamyl methoxylcinnamate, homosalate, para-aminobenzoic acid (PABA), octyl dimethyl PABA, 3-methylbenzylidene camphor, 4-methylbenzylidene camphor, benzophenone-3 and/or benzophenone-4.

In this particular embodiment, the fat-soluble photoprotective system (a) and its solubilising system (b) are as described above, as is the weight ratio (a)/(b).

Advantageously, the weight ratio phenylene bis-diphenyltriazine (c)/fat-soluble photoprotective system (a) is less than or equal to 0.35, in particular less than or equal to 0.33, advantageously less than or equal to 0.32, especially less than or equal to 0.30, and especially greater than or equal to 0.2.

Advantageously, the weight ratio phenylene bis-diphenyltriazine (c)/fat-soluble photoprotective system (a) is less than or equal to 0.35, in particular less than or equal to 0.33, advantageously less than or equal to 0.32, especially less than or equal to 0.30, and especially greater than or equal to 0.2, and the weight ratio fat-soluble photoprotective system (a)/C₁₂-C₁₅ alkyl benzoate is greater than or equal to 0.2, typically greater than or equal to 0.3, especially greater than or equal to 0.4, advantageously greater than or equal to 0.5, in particular strictly greater than 0.5 and especially less than 0.7, typically less than or equal to 0.6, in particular less than or equal to 0.55.

In a particular embodiment, the fat-soluble photoprotective system (a) correspond to one of the combinations of fat-soluble filters (a1)-(a8), which constitutes between 3 wt % and 19 wt %, especially between 5 wt % and 19 wt %, in particular between 7 wt % and 17 wt %, typically between 9 wt % and 15 wt %, for example between 10 wt % and 13 wt % of the composition relative to the total weight of the composition.

In particular, the fat-soluble photoprotective system (a) consists of the combination (a1).

In this particular embodiment, DHHB constitutes between 1 wt % and 10 wt %, for example between 3 wt % and 10 wt %, in particular between 4 wt % and 9 wt %, typically between 5 wt % and 8 wt %, for example between 5 wt % and 7 wt %, especially between 6 wt % and 7 wt % or advantageously between 5 wt % and 6 wt %, relative to the total weight of the composition, BEMT constitutes between 1 wt % and 4 wt %, especially between 2 wt % and 4 wt %, especially between 2 wt % and 3 wt % of the composition relative to the total weight of the composition, and EHT constitutes between 1 wt % and 6 wt %, typically between 2 wt % and 5 wt %, especially between 3 wt % and 5 wt %, in particular between 3.5 wt % and 4.5 wt % relative to the total weight of the composition.

In a particular embodiment, the composition according to the invention does not comprise diisopropyl sebacate, diethylhexyl succinate, isopropyl myristate and/or dibutyl adipate.

In another particular embodiment, the composition according to the invention does not comprise dicaprylyl carbonate, coco-caprylate, diisopropyl sebacate, diethylhexyl succinate, isopropyl palmitate, isopropyl myristate, propylheptyl caprylate, phenethyl benzoate, dibutyl adipate, diisopropyl adipate, glyceryl tri-2-ethylhexanoate, pentaerythrityl tetra-2-ethylhexanoate, cetyl 2-ethylhexanoate, isononyl isononanoate, butylene glycol dicaprylate/dicaprate, octyldodecanol, propylene glycol dicaprylate/dicaprate and/or dicaprylyl ether.

In a particular embodiment, the composition according to the invention does not comprise a water-soluble filter.

The absence of water-soluble filters in a composition according to the invention is particularly advantageous, in that water-soluble filters are, in general, more easily assimilated by marine organisms than fat-soluble or non-soluble filters. Consequently, the choice of the Applicant, consisting of using only fat-soluble filters or particulate filters, which are in particular hydro-dispersed, enables the impact of the composition according to the invention on the marine environment to be minimised.

In a particular embodiment, the composition according to the invention, comprises one or more particulate inorganic filters, such as titanium dioxide (TiO₂) or zinc oxide (ZnO).

The one or more particulate inorganic filters that are optionally present in the composition according to the invention, typically have a particle size D₅₀ between 15 nm and 150 nm.

In another particular embodiment, the composition according to the invention does not comprise any particulate inorganic filter.

In a particular embodiment, the total number of UV filters present in the composition is less than or equal to 5, preferably less than or equal to 4, and all the UV filters present in the composition constitute between 4 wt % and 25 wt %, in particular between 6 wt % and 20 wt %, especially between 7 wt % and 18 wt %, typically between 8 wt % and 17 wt %, relative to the total weight of the composition.

Advantageously, the composition according to the invention is photostable.

For the purposes of the present invention, “photostable” shall mean that after an irradiation of 5 MED and preferably 10 MED, a minimum of 80%, preferably a minimum of 85% and advantageously a minimum of 90% of the total SPF is preserved.

The term “SPF” designates the sun protection factor, the total SPF being evaluated for wavelengths between 290 and 400 nm.

Its value will be determined according to the method described in the examples of the present description recommended by COLIPA (European trade association for cosmetic and personal care industry), in its guidelines published in March 2011.

In a particular embodiment, the SPF of a composition according to the invention is greater than or equal to 50, advantageously greater than or equal to 60.

In another particular embodiment, the ratio SPF/UVA is less than or equal to 3, in accordance with the regulations in force. The denominator of said quotient corresponds to the UVA part (320 to 400 nm) of the total SPF, and can be calculated according to the method recommended by COLIPA (European trade association for cosmetic and personal care industry), in its guidelines published in March 2011. Advantageously, the ratio SPF/UVA is less than or equal to 2.5.

The composition according to the invention can further comprise one or more cosmetically or dermatological acceptable excipients.

In the present invention, the term “cosmetically or dermatologically acceptable” means that which is usable in the preparation of a cosmetic or dermatological composition, which is generally safe, non-toxic and neither biologically nor otherwise undesirable and which is acceptable for human cosmetic or dermatological use.

The term “cosmetically or dermatologically acceptable excipient” shall mean any cosmetically or dermatologically acceptable adjuvant enabling the manufacture, preservation or administration of the composition.

The compositions according to the invention may thus comprise conventional pharmaceutical or dermatological adjuvants chosen, in particular, from emulsifiers, consistency agents, water retention agents, texture agents, opacifiers, colouring pigments, anti-foaming agents, perfumes, preservatives, polymers, fillers, sequestering agents, bactericides, odour absorbers, alkalising or acidifying agents, surfactants, free radical scavengers, vitamins E and C, alpha-hydroxyacids, active agents (in particular softeners, moisturisers, antioxidants) or any other ingredient normally used for the manufacture of sun-protection compositions.

The composition may further comprise a polyol that is miscible with water at ambient temperature (approximately 25° C.), in particular chosen from polyols having, in particular, 2 to 20 carbon atoms, preferably having 2 to 10 carbon atoms, and more preferably having 2 to 6 carbon atoms, such as glycerine; glycol derivatives such as propylene glycol, butylene glycol, pentylene glycol, hexylene glycol, dipropylene glycol, diethylene glycol; glycol ethers, such as C₁-C₄ alkyl ethers of mono-, di- or tri-propylene glycol, C₁-C₄ alkyl ethers of mono-, di- or tri-ethylene glycol and the mixtures thereof.

The composition may also comprise one or more anionic, cationic and non-ionic emulsifiers chosen from the derivatives of glucose, such as cetearyl glucoside, arachidyl glucoside, lauryl glucoside, polyglyceryl-3 methylglucose distearate and the methyl glucose sesquistearate; sucrose derivatives, such as the sucrose polystearate and sucrose palmitate; fatty acid glycerides, such as glyceryl stearate, glyceryl oleate and glyceryl stearate citrate; glutamic acid derivatives, such as sodium stearoyl glutamate; sulfosuccinic acid derivatives such as the disodium cetearyl sulfosuccinate and the sodium dioctyl sulfosuccinate; phosphoric acid derivatives, such as potassium cetyl phosphate; polyglyceryl fatty acid esters, such as the polyglyceryl-3-diisostearate and polyglyceryl-2-dipolyhydroxystearate; sorbitol derivatives, such as polysorbate-n, PEG-10 sorbitan laurate and the sorbitan esters; fatty alcohol and fatty acid polyglycolethers, such as ceteareth-20, beheneth-25, steareth-2, trideceth-9, PEG-5 ethylhexanoate and PEG-100 stearate; sodium lauryl sulfate, sodium stearate, and the copolymers and derivatives of organo-modified oxyalkenylated silicone/polysiloxane/polyalkyl/polyether.

In a particular embodiment, the one or more emulsifiers constitute between 1 wt % and 5 wt %, preferably between 1 wt % and 3 wt %, relative to the total weight of the composition.

In a particular embodiment, the composition according to the invention comprises one or more emulsifiers chosen from potassium cetyl phosphate, lauryl glucoside and glyceryl stearate citrate.

In a particular embodiment, the composition according to the invention does not comprise an ethoxylated emulsifier and/or an emulsifier comprising a PEG chain.

In particular, the composition does not comprise sorbitol derivatives, such as polysorbate-n and the PEG-10 sorbitan laurate; fatty alcohol and fatty acid polyglycolethers, such as ceteareth-20, beheneth-25, steareth-2 and PEG-100 stearate; and copolymers and derivatives of organo-modified oxyalkenylated silicone/polysiloxane/polyalkyl/polyether.

The composition may also comprise one or more solid consistency agents, chosen from fatty alcohols, such as cetyl alcohol, cetearyl alcohol and stearyl alcohol; fatty acids such as stearic acid; fatty acid esters, such as myristyl stearate; polysaccharides or derivatives, such as xanthan gum, guar gum, agar gum, alginates, gellan and carrageenan gum; cellulose derivatives, such as hydroxypropylcellulose; waxes such as beeswax, carnauba wax, microcrystalline wax, ceresin, and ozocerite; polyacrylates or acrylic acid or cross-linked polyacrylamide homopolymers, such as carbomers, acrylate copolymers, cross-linked acrylate/(C₁₀-C₃₀)alkylacrylate copolymers, behenethyl-25 acrylate/methacrylate; and the derivatives of silicates such as magnesium silicates.

In a particular embodiment, the one or more consistency agents constitute between 0.1 wt % and 2 wt %, preferably between 0.1 wt % and 1 wt %, relative to the total weight of the composition.

In a particular embodiment, the composition according to the invention comprises one or more consistency agents chosen from the fatty alcohols such as cetyl alcohol, cetearyl alcohol and stearyl alcohol; fatty acids such as stearic acid; fatty acid esters such as myristyl stearate; polysaccharides or derivatives such as the xanthan gum, guar gum, agar gum, alginates, gellan gum and carrageenan gum; and cellulose derivatives such as hydroxypropyl cellulose.

In a particular embodiment, the composition according to the invention does not comprise a consistency agent chosen from the acrylates and their derivatives.

The composition may also comprise one or more water retention agents, chosen from a copolymer of N-vinylpyrrolidone (VP) and eicosen (INCI: VP/eicosen copolymer), a cross-linked trimethylpentanediol polymer, adipic acid and glycerol (INCI: trimethylpentanediol/adipic acid/glycerin crosspolymer), polyamide 3 (INCI), a copolymer of acrylate and octylacrylamide (INCI: acrylates/octylacrylamide copolymer), and triacontanyl PVP (INCI).

In a particular embodiment, the one or more water retention elements constitute between 0 wt % and 2 wt %, preferably between 0 wt % and 1 wt %, relative to the total weight of the composition.

Preferably, the composition according to the invention comprises VP/eicosen copolymer as a water retention agent.

The composition may also comprise one or more texture agents in powder form, in particular chosen from the ingredients of the starch family, such as rice starch, corn starch and tapioca starch; talcs and cellulose derivatives such as microcrystalline cellulose.

In a particular embodiment, the one or more texture agents constitute between 1 wt % and 10 wt %, preferably between 1 wt % and 5 wt %, relative to the total weight of the composition.

In a particular embodiment, the composition according to the invention does not comprise any texture agent chosen from the derivatives of nylon and poly(methyl methacrylate) (PMMA).

The composition may also comprise colouring pigments, in particular chosen from C177891, C177491, C177492 and C177499 (INCI names).

In a particular embodiment, the composition according to the invention comprises a quantity of C₁-C₄ alcohols, such as ethanol and isopropanol, less than or equal to 4 wt %, preferably less than or equal to 2 wt %, especially less than or equal to 1 wt %, in particular less than or equal to 0.5 wt %, relative to the total weight of the composition.

In another particular embodiment, the composition according to the invention contains no ethanol or isopropanol.

In a particular embodiment, the composition according to the invention comprises a quantity of volatile ingredients less than or equal to 5 wt %, preferably less than or equal to 2 wt %, especially less than or equal to 1 wt %, in particular less than or equal to 0.5 wt %, relative to the total weight of the composition.

For the purposes of the present invention, “volatile ingredients” shall mean non-alcoholic, volatile organic or inorganic compounds, such as volatile esters, silicones or alkanes, characterised by a flash point less than 60° C.

For the purposes of the present invention, “flash point” (denoted FP), shall mean the minimum temperature at which a combustible substance gives of vapours in a concentration sufficient to form a gaseous mixture with the ambient air which ignites on contact with a flame or a hot spot, but insufficient for the combustion to propagate by itself in the absence of the so-called pilot flame. According to regulation (EC) No 1272/2008 relating to the classification, labelling and packaging of substances and mixtures, a substance having a FP such that 23° C.≤FP<60° C. belongs to category 3 “low flammability”.

The volatile esters can include, in particular, isohexyl neopentanoate.

The volatile silicones can include, in particular, cyclopentasiloxane.

The volatile alkanes can include, in particular, isohexadecane.

In another particular embodiment, the composition according to the invention contains no volatile ingredients within the meaning of the present invention.

The composition may also comprise alkalising or acidifying agents, and more particularly acids and bases which can adjust the pH range of said composition. The bases can be mineral (soda, potash, ammonia, etc.) or organic, such as mono-, di- or triethanolamine, an aminomethylpropanediol, N-methyl-glucamine, basic amino acids such as arginine and lysine, and the mixtures thereof. The acids may be mineral (hydrochloric acid, etc.) or organic, such as lactic acid and citric acid.

The compositions according to the invention may further comprise additional active agents chosen, in particular, from hydrating agents, desquamating agents, barrier function improving agents, depigmentation agents, antioxidants, dermocontracting agents, antiglycation agents, agents stimulating the synthesis of dermal and/or epidermal macromolecules and/or preventing their degradation, agents stimulating the proliferation of fibroblasts or of keratinocytes and/or the differentiation of keratinocytes, NO synthase inhibitors, agents increasing sebaceous gland activity, tensing agents, liporestructuring agents, slimming agents, agents promoting cutaneous microcirculation, soothing and/or irritating agents, sebum regulators or anti-seborrhoeic agents, astringent agents, heating agents, anti-inflammatory agents, anti-acne agents, and the mixtures thereof.

In a particular embodiment, the composition according to the invention contains no silicone.

The composition according to the invention is more particularly a sun-protection composition, commonly called a sun-care composition, for protecting the skin (face and/or body) and/or hair against ultraviolet radiation.

Another object of the present invention is a method for protecting the skin (face and/or body) and/or hair against ultraviolet radiation, comprising the application on the skin (face and/or body) and/or on the hair of an above-described composition.

The present invention further relates to a composition as described above for use thereof for protecting the skin (face and/or body) and/or hair against ultraviolet radiation.

The present invention further relates to the use of a composition as described above for protecting the skin (face and/or body) and/or hair against ultraviolet radiation.

The invention further relates to a method for preparing a composition according to the invention as described above, in the form of an oil-in-water emulsion, comprising the following steps:
solubilising the system of one or more fat-soluble filters in the solubilising system (b) at a temperature between 70° C. and 85° C., typically at 80° C., while stirring, preferably while stirring mechanically:
adding other constituent ingredients of the oily phase of the emulsion to the mixture 30 previously obtained, at a temperature between 70° C. and 85° C., typically at 80° C., while stirring, preferably while stirring mechanically;
dispersing the one or more particulate organic filters (c) in water, at a temperature between 70° C. and 85° C., typically at 80° C., while stirring, preferably while stirring mechanically;
adding other constituent ingredients of the aqueous phase of the emulsion to the mixture previously obtained, at a temperature between 70° C. and 85° C., typically at 80° C., while stirring, preferably while stirring mechanically;
introducing the previously obtained oily phase into the previously obtained aqueous phase, while stirring, preferably while stirring mechanically, in order to obtain a homogeneous solution;
cooling the emulsion thus obtained to ambient temperature; and
optionally, adding perfume, active agents and/or texture agents in the form of powders, while stirring, preferably while stirring mechanically.

### Examples

The following abbreviations have been used:

| | |
|---|---|
| MED | minimum erythemal dose |
| SPF | sun protection factor |
| MTT | 3-(4,5-dimethylthiazol-2-yl)-2,5-diphenyltetrazolium bromide |
| PEG | polyethylene glycol |
| PMMA | poly(methyl methacrylate) |
| UV | ultraviolet radiation |
| VP | N-vinylpyrrolidone |

I. Solubilising System

The ability of three combinations S1, S2 and S3 of liquid lipophilic emollients to solubilise the same fat-soluble photoprotective system; in this case, the system (a1) as described above, has been evaluated according to the following protocol:
solubilising of the fat-soluble photoprotective system (a1) at 80° C. while stirring, preferably while stirring mechanically by means of a magnetic bar, in the combination of lipophilic emollients;
cooling to ambient temperature;
storing a sample of the solution obtained at ambient temperature, and cooling another sample to 5° C., for storing likewise;
regularly over time, visually observing the appearance of any recrystallisation.

The results obtained for the combinations S1, S2 and S3 are reported in the table below, in which the quantity of each lipophilic emollient is expressed as a percentage by weight relative to the total weight of the combination 51, S2 or S3, and the quantity of each fat-soluble filter is expressed as a percentage by weight relative to the total weight of the fat-soluble photoprotective system (a1).

| Fat-soluble filters (% wt/wt) | Fat-soluble photoprotective system (a1) | | |
|---|---|---|---|
| BEMT | 20-25 | | |
| EHT | 30-35 | | |
| DHHB | 45-50 | | |
| Lipophilic | | | |
| emollients (% wt/wt) | S1 | S2 | S3 |
| dicaprylyl carbonate | — | 39 | 36 |
| caprylic/capric triglycerides | — | 9 | 16 |
| C₁₂-C₁₅ alkyl benzoate | 100 | 52 | 4 |
| Ratio | 0.61 | 0.53 | 0.48 |
| fat-soluble photoprotective | | | |
| system (a1)/lipophilic | | | |
| emollients | | | |

Visual observation

| | | | |
|---|---|---|---|
| 24 hours at ambient | No crystals | No crystals | No crystals |
| temperature | | | |
| 1 month at 5° C. | No crystals | Some | Some |
| | | crystals | crystals |

Only 51 constitutes a solubilising system according to the present invention, which enables, on the one hand, the fat-soluble photoprotective system to be solubilised so as to obtain a homogeneous clear solution and, on the other hand, recrystallisation of the fat-soluble UV filters to be prevented over time.

By way of comparison, the stability of another oily phase of a sun protection emulsion, corresponding to that of example B3 of application WO 2016/012586, has been evaluated according to the following protocol:
solubilising the fat-soluble photoprotective system at 80° C. while stirring mechanically by means of a magnetic bar, in the solubilising system;
cooling to ambient temperature;
storing the solution obtained at ambient temperature;
regularly over time, visually observing the appearance of any recrystallisation.

Fat-soluble photoprotective system (a)

| | |
|---|---|
| BMDBM | 5 g, i.e. 23% (wt/wt) |
| DHHB | 16.7 g, i.e. 77% (wt/wt) |

Solubilising system (b)

| | |
|---|---|
| C₁₂-C₁₅ alkyl benzoate | 13.3 g (100%) |
| Ratio (a)/(b) | 1.63 |

Visual observation

| | |
|---|---|
| 24 hours at ambient temperature | No crystals |
| 15 days at ambient temperature | Very many crystals |

II. Compositions

In the following formulas, 5,6,5′,6′-tetraphenyl-3,3′-(1,4-phenylene)-bis[1,2,4]triazine is in the form of an aqueous dispersion comprising 45 wt % (wt/wt) of active material and is characterised by a D₅₀ between 150 and 250 nm.

II.1. Method for Preparing Emulsions

The ingredients of the oily phase (in particular the one more fat-soluble UV filters, the solubilising system, the one or more emulsifiers, the one or more fat-soluble consistency agents on the one hand, and the ingredients of the aqueous phase (in particular one or more particulate organic filters, the one or more polyols, the one or more water-soluble consistency agents, and water), on the other hand, are respectively mixed and heated to 80° C. while stirring.

Then the oily phase is added to the aqueous phase while stirring until homogenisation. Finally, the composition thus obtained is cooled to ambient temperature while stirring.

Optionally, the perfume, active agents and the one or more texture agents, in the form of powder, are added while stirring, after cooling of the final composition.

II.2. Formula 1

| | |
|---|---|
| Ingredients | % (wt/wt) |

Fat-soluble photoprotective system

| | |
|---|---|
| Bis-ethylhexyloxyphenol methoxyphenyl triazine | 2-3 |
| Diethylamino hydroxybenzoyl hexyl benzoate | 5-7 |
| Ethyl hexyl triazone | 3-4 |

Solubilising system

| | |
|---|---|
| C₁₂-C₁₅ alkyl benzoate | 20-25 |
| Particulate organic filter | |
| 5,6,5′,6′-tetraphenyl-3,3′-(1,4-phenylene)-bis[1,2,4]triazine | 3-4 |

Other components

| | |
|---|---|
| Glycerine | 4.0 |
| Demineralised water | QS for 100 |
| Xanthan gum | 0.2 |
| Preservatives | QS |
| Perfume | QS |
| Stearyl alcohol | 1.5 |
| Glycerol monostearate | 2.0 |
| VP/eicosen copolymer | 1.0 |
| Rice starch | 2-5 |
| Potassium cetyl phosphate | 2.0 |

The fat-soluble photoprotective system advantageously constitutes between 12 wt % and 14 wt % relative to the total weight of the composition.

II.3. Formula 2

| | |
|---|---|
| Ingredients | % (wt/wt) |

Fat-soluble photoprotective system

| | |
|---|---|
| Bis-ethylhexyloxyphenol methoxyphenyl triazine | 2-3 |
| Diethylamino hydroxybenzoyl hexyl benzoate | 5-7 |
| Ethyl hexyl triazone | 3-4 |

Solubilising system

| | |
|---|---|
| C₁₂-C₁₅ alkyl benzoate | 20-25 |

Particulate organic filter

| | |
|---|---|
| 5,6,5′,6′-tetraphenyl-3,3′-(1,4-phenylene)-bis[1,2,4]triazine | 3-4 |

Other components

| | |
|---|---|
| Glycerine | 3.0 |
| Demineralised water | QS for 100 |
| Xanthan gum | 0.3 |
| Preservatives | QS |
| Perfume | QS |
| Stearyl alcohol | 1.0 |
| Glycerol monostearate | 1.0 |
| VP/eicosen copolymer | 1.0 |
| Rice starch | 2-5 |
| Potassium cetyl phosphate | 2.0 |

The fat-soluble photoprotective system advantageously constitutes between 12 wt % and 14 wt % relative to the total weight of the composition.

II.4. Formula 3

| | |
|---|---|
| Ingredients | % (wt/wt) |

Fat-soluble photoprotective system

| | |
|---|---|
| Bis-ethylhexyloxyphenol methoxyphenyl triazine | 2-3 |
| Diethylamino hydroxybenzoyl hexyl benzoate | 5-7 |
| Ethyl hexyl triazone | 3-4 |

Solubilising system

| | |
|---|---|
| C₁₂-C₁₅ alkyl benzoate | 20-25 |
| Caprylic/capric triglycerides | 0.2 |

Particulate organic filter

| | |
|---|---|
| Methylene bis-benzotriazolyl tetramethylbutylphenol | 2-4 |

Other components

| | |
|---|---|
| Glycerine | 4.0 |
| Demineralised water | QS for 100 |
| Xanthan gum | 0.2 |
| Preservatives | QS |
| Perfume | QS |
| Stearyl alcohol | 1.5 |
| Glycerol monostearate | 2.0 |
| VP/eicosen copolymer | 1.0 |
| Potassium cetyl phosphate | 2.0 |

II.5. Formula 4

| | |
|---|---|
| Ingredients | % (wt/wt) |

Fat-soluble photoprotective system

| | |
|---|---|
| Bis-ethylhexyloxyphenol methoxyphenyl triazine | 2-3 |
| Diethylamino hydroxybenzoyl hexyl benzoate | 6-7 |
| Diethylhexyl butamido triazone | 3-4 |

Solubilising system

| | |
|---|---|
| C₁₂-C₁₅ alkyl benzoate | 20-25 |

Particulate organic filter

| | |
|---|---|
| 5,6,5′,6′-tetraphenyl-3,3′-(1,4-phenylene)-bis[1,2,4]triazine | 3-4 |

Other components

| | |
|---|---|
| Glycerine | 4.0 |
| Demineralised water | QS for 100 |
| Xanthan gum | 0.2 |
| Preservatives | QS |
| Cetyl alcohol | 1.5 |
| Glycerol monostearate | 2.0 |
| VP/eicosen copolymer | 1.0 |
| Lauryl glucoside | 5.0 |

II.6. Formula 5

| | |
|---|---|
| Ingredients | % (wt/wt) |

Fat-soluble photoprotective system

| | |
|---|---|
| Bis-ethylhexyloxyphenol methoxyphenyl triazine | 2-3 |
| Butyl methoxydibenzoylmethane | 3-4 |
| Ethyl hexyl triazone | 3-4 |

Solubilising system

| | |
|---|---|
| C₁₂-C₁₅ alkyl benzoate | 20-25 |

Particulate organic filter

| | |
|---|---|
| 5,6,5′,6′-tetraphenyl-3,3′-(1,4-phenylene)-bis[1,2,4]triazine | 3-4 |

Other components

| | |
|---|---|
| Glycerine | 4.0 |
| Demineralised water | QS for 100 |
| Xanthan gum | 0.2 |
| Preservatives | QS |
| Perfume | QS |
| Stearyl alcohol | 1.5 |
| Glycerol monostearate | 2.0 |
| VP/eicosen copolymer | 1.0 |
| Rice starch | 2-5 |
| Potassium cetyl phosphate | 2.0 |

II.7. Formula 6

| | |
|---|---|
| Ingredients | % (wt/wt) |

Fat-soluble photoprotective system

| | |
|---|---|
| Diethylamino hydroxybenzoyl hexyl benzoate | 5-6 |
| Ethyl hexyl triazone | 3-4 |

Solubilising system

| | |
|---|---|
| C₁₂-C₁₅ alkyl benzoate | 20-25 |

Particulate organic filter

| | |
|---|---|
| 5,6,5′,6′-tetraphenyl-3,3′-(1,4-phenylene)-bis[1,2,4]triazine | 3-4 |
| methylene bis-benzotriazolyl tetramethylbutylphenol | 5-7 |

Other components

| | |
|---|---|
| Glycerine | 4.0 |
| Demineralised water | QS for 100 |
| Xanthan gum | 0.2 |
| Preservatives | QS |
| Perfume | QS |
| Stearyl alcohol | 1.5 |
| Glycerol monostearate | 2.0 |
| VP/eicosen copolymer | 1.0 |
| Microcrystalline cellulose | 2-5 |
| Potassium cetyl phosphate | 2.0 |

### II.8. Formula 7

| | |
|---|---|
| Ingredients | % (wt/wt) |

Fat-soluble photoprotective system

| | |
|---|---|
| Bis-ethylhexyloxyphenol methoxyphenyl triazine | 3-4 |
| Diethylamino hydroxybenzoyl hexyl benzoate | 5-6 |

Solubilising system

| | |
|---|---|
| C₁₂-C₁₅ alkyl benzoate | 20-25 |

Particulate organic filter

| | |
|---|---|
| 5,6,5′,6′-tetraphenyl-3,3′-(1,4-phenylene)-bis[1,2,4]triazine | 3-4 |

Particulate mineral filter

| | |
|---|---|
| TiO₂ | 4-5 |

Other components

| | |
|---|---|
| Glycerine | 4.0 |
| Demineralised water | QS for 100 |
| hydroxypropylcellulose | 0.2 |
| Preservatives | QS |
| Perfume | QS |
| Stearyl alcohol | 1.5 |
| Glycerol monostearate | 2.0 |
| VP/eicosen copolymer | 1.0 |
| Corn starch | 2-5 |
| Potassium cetyl phosphate | 2.0 |

III. Determination of the particle size of 5,6,5′,6′-tetraphenyl-3,3′-(1,4-phenylene)-bis[1,2,4]triazine

The D₅₀ of particles of phenylene bis-diphenyltriazine is determined using a Mastersizer 3000 laser diffraction particle size analyser, by the liquid method.

A sample of an aqueous dispersion of phenylene bis-diphenyltriazine is placed under magnetic stirring (2500 rpm) for 2 minutes.

1 mL of the sample is taken and introduced into a flask that is then filled to 100 mL with ultrapure water.

The solution thus obtained is placed under magnetic stirring (2500 rpm) for 30 seconds. The solution to be examined is taken and introduced into the measuring vessel up to the 15% obscuration limit.

Three independent tests are carried out and three independent measurements (red laser: background noise measurement, 10 seconds, sample measurement, 10 seconds; blue laser: background noise measurement, 10 seconds, sample measurement, 10 seconds) are carried out per test.

The results are obtained in volume, and then converted into a number using the software.

IV. Evaluation of the Sun Protection

The evaluation of the parameters allowing quantification of the photo-protection provided by a composition according to the invention is carried out according to the methods recommended by COLIPA (European trade association for cosmetic and personal care industry), in its guidelines published in March 2011.

IV.1. Equipment

The UV spectrometer

The spectrophotometer measures the spectral transmittance through a plate with or without a layer of sun-protection composition on its surface.

The spectrophotometer must enable measurements between 290 nm and 400 nm. In order to reduce the variability between the measurement readings and to compensate for the lack of uniformity in the layer of product, it is recommended that the area of the reading zones is at least 0.5 cm².

The spectrophotometer used for these measurements is a Labsphere® UV-1000S or 20005.

The Plate

The plate is the material on which the sun-protection composition is applied. This material must be transparent to UV, non-fluorescent, photostable and inert with respect to the components of the tested compositions. For this protocol, PMMA plates proved ideal.

UV Source

The UV source is a solar simulator with a xenon arc lamp giving out a Visible+UVA+UVB spectrum. The UV source used for this study is a Suntest CPS+(Atlas).

IV.2. Method for Determining the SPF and Photostability

Measurements of the Transmission Through an Untreated Plate

In a first step, it is necessary to determine the UV transmission through the control plate. This is prepared by spreading several microlitres of glycerine in such a way that the surface of the plate is entirely covered.

Application of the Sample

The sample to be tested is applied on the PMMA plate with a dose of 1.3 mg/cm² (actual quantity remaining on the plate). In order to guarantee the accuracy of the dose and the reproducibility of results, the application zone is greater than 10 cm².

The sample to be tested is applied in the form of a large number of small drops of the same volume, distributed over the entire surface of the plate.

In order to ensure that the quantity of product is correct, a method for checking the quantity of product applied must be adopted (for example: weighing the plate before after application of the product).

After application of the defined quantity of the sample, the sample must be spread over the entire plate as rapidly as possible (less than 30 seconds).

The sample is then placed in the dark at ambient temperature for 15 minutes, in order to promote the formation of a homogeneous film.

Measurement of Transmission Through a Plate Treated with the Sample

The plate treated with the sample is analysed using the spectrophotometer and the mean transmission value of UV radiation through the sample is determined for each wavelength from 290 nm to 400 nm (using the monochromatic absorbance data measured on the various zones of the plate).

Number of Measurements

At least three PMMA plates must be prepared for each sample. Each plate must be measured in at least nine different regions, unless almost the entire surface of the plate is measured by spectrophotometry.

Calculation of SPF In Vitro (Sun Protection Factor)

This is carried out using absorbance data A(λ) before and after irradiation with doses of 5 and 10 MED, according to the following formula:

$$SPF in vitro = \frac{{\int }_{λ = 290 nm}^{λ = 400 nm} E ( λ ) \cdot S ( λ ) \cdot d λ}{{\int }_{λ = 290 nm}^{λ = 400 nm} E ( λ ) \cdot S ( λ ) \cdot {10}^{- A ( λ )} d λ}$$

in which:
E(λ) corresponds to the erythemal efficiency spectrum,
S(λ) corresponds to the solar spectral irradiance,
A(λ) corresponds to the absorbance of the sample,
dλ is the variation in wavelength (1 nm).

Calculation of Photostability

$$Total UV photostability = \frac{SPF after irradiation}{SPF before irradiation} \times 100$$

The compositions according to the invention can be classified according to the categories indicated below, as a function of their photostability.

| | % of the total SPF conserved |
|---|---|
| Category | after irradiation of 5 MED |
| A | ≥90% |
| B | 85%-<90% |
| C | 80%-<85% |
| D | ≤80% |

IV.3. Results

The following results were obtained for formulas 1 and 2:

| | Formula 1 | Formula 2 |
|---|---|---|
| SPF in vitro | >70 | >70 |
| Ratio SPF/UVA | <3 | <3 |
| Total photostability | Category | Category |
| after irradiation at 5 MED | A | A |

V. Evaluation of Ocular Tolerance

V.1. In Vitro Model on Reconstructed Human Cornea Epithelium (HCE)

This test is an alternative method to animal experimentation for evaluating the ocular irritant potential on reconstructed human cornea epithelium.

The principle is based on determining the mean cytotoxicity index over 24 hours by application of the products on reconstructed human cornea epithelia.

Cell viability is studied by reducing MTT incorporated in the living cells to a tetrazolium salt which forms violet crystals that can be measured by colorimetry.

The parameter calculated is the MCl₂₄ₕ (“mean cytotoxicity index” over 24 hours) i.e. the mean mortality caused per hour. This makes it possible to determine whether or not the product is an irritant.

The method is described in the article of Doucet et al., Toxicology in vitro, 2006, 20, 499-512.

The compositions according to the invention can be classified according to the scoring system indicated below, as a function of their MCl₂₄ₕ.

| Category | MCl_{24 h} |
|---|---|
| 4: non-irritant | <4 |
| 3: slightly irritant | 4-<7 |
| 2: medium irritant | 7-<17 |
| 1: very irritant | ≥17 |

V.2. “Neutral Red Release” Method (NRR)

This test is an alternative method to animal experimentation for evaluating the ocular irritant potential.

The principle is based on evaluating the cytotoxicity by determining the concentration causing 50% mortality (CL50) using the neutral red release method on rabbit cornea fibroblasts from the SIRC after a contact time of 30 or 60 seconds.

The method is described in the decree of 27 Dec. 1999, published in the French Official Journal of 30 Dec. 1999.

After incubating the neutral red staining solution with the cells in culture on the plate, each product dilution is placed in contact with the cells for 60 seconds or 30 seconds, before being removed by rinsing. The viability of the cells is evaluated by spectrophotometry after addition of the release solution.

The cytotoxicity of the product is given by a scale described in the French Official Journal of 30 Dec. 1999, comprising 4 categories:

| Category | Cytotoxicity |
|---|---|
| 4 | negligible |
| 3 | unimportant |
| 2 | moderate |
| 1 | important |

V.3. Results

The following result were obtained for formula 1:

| | Formula 1 |
|---|---|
| HCE model | Rating 3 |
| NRR method | Rating 4 |

VI. Sensory Evaluation of a Composition According to the Invention by a Consumer Usage Test

The aim of this test is to characterise the perceptions and feelings during use of a composition according to the invention, and to determine the strengths and weaknesses under actual conditions of use.

78 consumers were recruited in France, aged from 18 to 65 years old (average 42 years old), and 84% of them have sensitive skin.

These consumers have evaluated the product corresponding to formula 2, at home.

The product was evaluated anonymously over a period of use of 14 days, with an evaluation required at the time of application, after 1 day and after 14 days.

It should be noted that no intolerance leading to stopping of the test was observed.

The following use protocol was given to the consumers.

Before going out, apply the composition on the neck and face in place of the usual sun-care product.

Reapply frequently, especially after sweating, swimming or wiping the skin.

| | | | | Imper- | Invisible |
|---|---|---|---|---|---|
| | Non- | Non- | Ease of | ceptible | on the |
| Criterion | greasy | sticky | application | finish | skin |
| Average score (/10) | 7.5 | 7.64 | 8.08 | 7.36 | 7.96 |
| % consumers | 77% | 77% | 82% | 73% | 86% |
| having attributed a | | | | | |
| score between 7 | | | | | |
| and 10 | | | | | |

The sun-care product composition according to the invention does indeed have an optimum sensory feel, which is especially manifest during the usage test by a product that it easy to spread and which, once applied, does not give the skin a greasy or sticky appearance. A very good perception of the consumers to the touch and visually is also to be noted.

1. A cosmetic or dermatological composition, in the form of an emulsion, comprising:
(a) a fat-soluble photoprotective system, consisting of one or more fat-soluble UV filters chosen from:
(i) hexyl 2-[4-(diethylamino)-2-hydroxybenzoyl]benzoate,
(ii) 2,4-bis[4-(2-ethylhexyloxy)-2-hydroxyphenyl]-6-(4-methoxyphenyl)-1,3,5-triazine,
(iii) butyl methoxydibenzoylmethane,
(iv) ethylhexyl triazone, and
(v) diethylhexyl butamido triazone;
(b) a solubilising system of the fat-soluble photoprotective system (a) comprising C₁₂-C₁₅ alkyl benzoate in a quantity greater than or equal to 80 wt % relative to the total weight of the solubilising system; and
(c) one or more particulate organic UV filters, chosen from:
(vi) 5,6,5′,6′-tetraphenyl-3,3′-(1,4-phenylene)-bis[1,2,4]triazine,
(vii) methylene bis-benzotriazolyl tetramethylbutylphenol, and
(viii) tris-biphenyl triazine;
wherein the composition comprises from 3 wt % to 19 wt % of the fat-soluble photoprotective system (a) relative to the total weight of the composition; and
wherein the composition comprises from 15 wt % to 30 wt % of the solubilising system (b) relative to the total weight of the composition;
wherein the composition does not comprise ethylhexyl salicylate, octocrylene, ethylhexyl methoxycinnamate, isoamyl methoxylcinnamate, homosalate, para-aminobenzoic acid (PABA), octyl dimethyl PABA, 3-methylbenzylidene camphor, 4-methylbenzylidene camphor, benzophenone-3 and benzophenone-4.

2. The composition according to claim 1, wherein the solubilising system (b) is formed from one or more lipophilic emollients chosen from C₁₂-C₁₅ alkyl benzoate, caprylic/capric triglycerides, dicaprylyl carbonate, coco caprylate, diisopropyl sebacate, diethylhexyl succinate, isopropyl palmitate, isopropyl myristate, propylheptyl caprylate, phenethyl benzoate, dibutyl adipate, diisopropyl adipate, glyceryl tri-2-ethylhexanoate, pentaerythrityl tetra-2-ethylhexanoate, cetyl 2-ethylhexanoate, isononyl isononanoate, butylene glycol dicaprylate/dicaprate, octyldodecanol, propylene glycol dicaprylate/dicaprate and dicaprylyl ether.

3. The composition according to claim 1, comprising from 1 wt % to 8 wt % of the one or more particulate organic UV filters (c) relative to the total weight of the composition.

4. The composition according to claim 1, wherein the fat-soluble photoprotective system (a) consists of one of following combinations of fat-soluble filters:
(a1) hexyl 2-[4-(diethylamino)-2-hydroxybenzoyl]benzoate, 2,4-bis[4-(2-ethylhexyloxy)-2-hydroxyphenyl]-6-(4-methoxyphenyl)-1,3,5-triazine and ethylhexyl triazone;
(a2) hexyl 2-[4-(diethylamino)-2-hydroxybenzoyl]benzoate, 2,4-bis[4-(2-ethylhexyloxy)-2-hydroxyphenyl]-6-(4-methoxyphenyl)-1,3,5-triazine and diethylhexyl butamido triazone;
(a3) 2,4-bis[4-(2-ethylhexyloxy)-2-hydroxyphenyl]-6-(4-methoxyphenyl)-1,3,5-triazine and ethylhexyl triazone;
(a4) 2,4-bis[4-(2-ethylhexyloxy)-2-hydroxyphenyl]-6-(4-methoxyphenyl)-1,3,5-triazine and diethylhexyl butamido triazone;
(a5) hexyl 2-[4-(diethylamino)-2-hydroxybenzoyl]benzoate, butyl methoxydibenzoylmethane, 2,4-bis[4-(2-ethylhexyloxy)-2-hydroxyphenyl]-6-(4-methoxyphenyl)-1,3,5-triazine and ethylhexyl triazone;
(a6) hexyl 2-[4-(diethylamino)-2-hydroxybenzoyl]benzoate, butyl methoxydibenzoylmethane, 2,4-bis[4-(2-ethylhexyloxy)-2-hydroxyphenyl]-6-(4-methoxyphenyl)-1,3,5-triazine and diethylhexyl butamido triazone;
(a7) butyl methoxydibenzoylmethane, 2,4-bis[4-(2-ethylhexyloxy)-2-hydroxyphenyl]-6-(4-methoxyphenyl)-1,3,5-triazine and ethylhexyl triazone; and
(a8) butyl methoxydibenzoylmethane, 2,4-bis[4-(2-ethylhexyloxy)-2-hydroxyphenyl]-6-(4-methoxyphenyl)-1,3,5-triazine and diethylhexyl butamido triazone.

5. The composition according to claim 4, wherein the fat-soluble photoprotective system (a) consists of the combination of fat-soluble filters (a1).

6. The composition according to claim 1, wherein the weight ratio between the fat-soluble photoprotective system (a) and the solubilising system (b) is greater than or equal to 0.5.

7. The composition according to claim 1, wherein the weight ratio between the fat-soluble photoprotective system (a) and the C₁₂-C₁₅ alkyl benzoate is greater than or equal to 0.5.

8. The composition according to claim 1, wherein the weight ratio between the one or more particulate organic UV filter(s) (c) and the fat-soluble photoprotective system (a) is less than or equal to 0.35.

9. The composition according to claim 1, comprising from 1 wt % to 4 wt % of 2,4-bis[4-(2-ethylhexyloxy)-2-hydroxyphenyl]-6-(4-methoxyphenyl)-1,3,5-triazine relative to the total weight of the composition.

10. The composition according to claim 1, comprising from 1 wt % to 10 wt % of hexyl 2-[4-(diethylamino)-2-hydroxybenzoyl]benzoate relative to the total weight of the composition.

11. The composition according to claim 1, comprising from 1 wt % to 6 wt % of ethylhexyl triazone relative to the total weight of the composition.

12. The composition according to claim 1, wherein the one or more particulate organic filters (c) consists of a single hydro-dispersed organic filter which is 5,6,5′,6′-tetraphenyl-3,3′-(1,4-phenylene)-bis[1,2,4]triazine.

13. The composition according to claim 1, wherein the total number of UV filters present in the composition is less than or equal to 5.

14. The composition according to claim 1, further comprising one or more C₁-C₄ alcohols in an amount of less than or equal to 4 wt % relative to the total weight of the composition or comprising no C₁-C₄ alcohol.

15. The composition according to claim 1, further comprising one or more particulate inorganic filters.

16. The composition according to claim 1, further comprising from 1 wt % to 10 wt % of one or more texture agents relative to the total weight of the composition,
wherein the one or more texture agents is in the form of a powder and is chosen from talcs and celluloses.

17. (canceled)

18. The composition according to claim 2, wherein the solubilising system (b) is formed from one or more lipophilic emollients chosen from C₁₂-C₁₅ alkyl benzoate, caprylic/capric triglycerides and dicaprylyl carbonate.

19. The composition according to claim 1, comprising from 2 wt % to 4 wt % of 2,4-bis[4-(2-ethylhexyloxy)-2-hydroxyphenyl]-6-(4-methoxyphenyl)-1,3,5-triazine, from 5 wt % to 7 wt % of hexyl 2-[4-(diethylamino)-2-hydroxybenzoyl]benzoate, and from 3 wt % and 5 wt % of ethylhexyl triazone relative to the total weight of the composition.

20. The composition according to claim 12, wherein the one or more particulate organic filters (c) has a particle size D₅₀ from 100 to 1000 nm.

21. The composition according to claim 12, comprising from 2 wt % to 5 wt % of the one or more particulate organic filters (c) relative to the total weight of the composition.

22. The composition according to claim 15, wherein the one or more particulate inorganic filters is selected from titanium dioxide (TiO₂), zinc oxide (ZnO) and a mixture thereof.

23. A method for protecting skin and/or hair against ultraviolet radiation comprising applying on the skin and/or the hair of a person in need thereof an effective amount of a composition according to claim 1.
